(19) United States
(12) Patent Application Publication
(10) Pub. No.: US 2005/0255190 A1
(43) Pub. Date: Nov. 17, 2005

(54) Method for processing cereal material

(76) Inventors: Suhas K. Mehra, Miamisburg, OH (US); Eugene J. Fox, Dayton, OH (US); Aharon M. Eyal, Jerusalem (IL); Michael R. Rizor, Cedar Rapids, IA (US); James T. Walsh II, Dayton, OH (US); Donald Lee Shandera, Ashland, NE (US); Ki Park, Dayton, OH (US); Mason Marsh, Fairborn, OH (US); Eugene M. Peters Jr, Kettering, OH (US)

Correspondence Address: CARGILL, INCORPORATED, LAW/24, 15407 MCGINTY ROAD WEST, WAYZATA, MN, 55391, US

(21) Appl. No.: 10/521,129

(22) PCT Filed: Jul. 22, 2003
(86) PCT No.: PCT/US03/22873

§ 371 Date: Jan. 12, 2005

Related U.S. Application Data
(60) Provisional application No. 60/398,030, filed on Jul. 23, 2002.

(51) Int. Cl. C12C 11/00; C12P 7/06

(52) U.S. Cl. 426/11; 435/161

(57) ABSTRACT

There is disclosed a method for processing cereal material that allows for a reduction of time required for steeping and a reduction in the number of unit operations in conventional corn wet milling. The method involves continuously and simultaneously having solvent absorbed by a cereal material and abrading the cereal material. There is disclosed also the use of products of the process in the production of fermentation feedstock. There is also disclosed also for the use fermentation feedstock.

This application claims priority from U.S. Provisional Application Ser. No. 60/398,030, filed Jul. 23, 2002, the entire contents of which are incorporated herein by reference.

### FIELD OF THE INVENTION

The present invention relates to a method for processing cereal material that allows for a reduction of time required for steeping and also a reduction in unit operations in comparison to conventional wet milling process.

### BACKGROUND OF THE INVENTION

Grains, including corn, oats, barley, rye, wheat, rice, and sorghum contain various concentrations of starches, proteins, fiber, and other nutrients. Often, it is desirable to process the grain to isolate various fractions or streams that are enriched in one of these components. Since these components are contained within the grain kernel, most processing methods begin with a step in which the kernel is treated to expose or release raw components.

As the sweetener, vegetable oil and alcohol industries become more competitive, cereal milling processes that are more efficient and exhibit less effluent discharge are desired. Traditionally, cereal such as corn, sorghum, wheat, rice and the like have been processed either by wet milling, dry milling or extrusion. Most corn processed in the United States, however, is subjected to the wet milling process.

Steeping, the initial operation in corn wet milling, commonly involves soaking the corn kernels in water under controlled conditions of temperature and sulfur dioxide (SO₂) combined with a lactic acid fermentation in a series of tanks for a prolonged period of time, for example about 20 to about 48 hours. These conditions are employed to promote diffusion of water and SO₂ into the corn kernels, which in turn softens the kernels and disrupts the starch-protein matrix. The softening of the kernels that occurs during steeping enhances separation of germ and fiber from the endosperm, while disruption of the starch-protein matrix aids in producing high quality starch.

Steeping is a semi-batch operation, which involves high investment and maintenance costs associated with steeping equipment, large floor area requirements, poor flexibility for production rate changes, as well as high consumption of energy. Furthermore, during the steeping process a portion of the carbohydrates is often lost due to the fermentation process and some of the proteins may also be dissolved.

It would be advantageous to have a method of processing cereal material, such as corn, that would allow for a reduction of time required for steeping and also reduces the number of unit operations.

### SUMMARY OF THE INVENTION

The present invention relates to a method for processing a cereal material that comprises providing a cereal material, and continuously and simultaneously both absorbing solvent by the cereal material and abrading the cereal material for a period of about at least 1 minute.

The present invention also relates to a method for the production of a starch product. Furthermore, the present invention also relates to the recovery of an oil containing germ product, a fiber product and/or a protein product.

The present invention also relates to a process for using the products resulting from the method of the invention in the production of a fermentation feedstock. The present invention also relates to a process for using the products resulting from the method of the invention as a fermentation feedstock.

### DETAILED DESCRIPTION OF THE INVENTION

The present invention relates to a method for processing a cereal material that comprises providing a cereal material, and continuously and simultaneously both absorbing solvent by the cereal material and abrading the cereal material for a period of about at least 1 minute.

The present invention also relates to a method for the production of a starch product. Furthermore, the present invention also relates to the recovery of an oil containing germ product, a fiber product and a protein product.

The present invention also relates to a process for using the products resulting from the method of the invention in the production of a fermentation feedstock. The present invention also relates to a process for using the products resulting from the method of the invention as a fermentation feedstock.

The products of the present method may be used for any conventional applications in addition to the use in the production of fermentation feedstock.

The term cereal material as used herein means any cereal material or portion thereof. In more detail, any cereal material can be utilized in the method described herein. Examples of suitable cereal materials are corn, sorghum, oats, barley, rye, wheat, rice or mixtures thereof In the present process the cereal material absorbs solvent. As solvent there may be used any aqueous or organic solvent or mixture thereof. Examples of organic solvents include hexane, isohexane, ethanol, methanol, propanol, isopropanol, butanol, acetone, dimethylformamide, dimethyl sulfoxide, and the like. Preferred for use is an aqueous solvent such as water containing sulfites, preferably introduced as sulfur dioxide and or salts of bisulfites. The absorption by the cereal material is achieved after a period of at least about 1 minute. Preferably the period of time is about 5 minutes to about 5 hours, and more preferably ranges from about 1 hour to about 3 hours.

Various additives may be incorporated into the solvents. For example, there may be incorporated additives that enhance the absorption of the solvent and/or separation of cereal material into components. Further, there may be incorporated reducing agents and/or pH adjusting agents; anti-foaming agents; wetting agents and the like. Examples of reducing agents include SO₂ and salts of sulfites and bisulfites, mercaptoethanol, thioglycolic acid and the like; suitable pH adjusting agents include lactic acid, acetic acid, hydrochloric acid, sodium hydroxide, lime and the like. Furthermore, enzymes such as cellulases, hemicellulases, proteases and the like may be utilized.

The abrading of the cereal material may be achieved in any known manner. Examples of suitable means for abrading cereal material are as follows: grind mills; pump impellers; agitators; blenders; attrition mills; roller mills; hammer mills; impact mills; pin mills and the like. Preferred examples include pump impellers, blenders, pin mills, agitators and grind mills.

The abrading of the cereal material may be carried out at any level of revolutions per minute preferably about 5 to about 10,000 revolutions per minute (rpm). Preferably the abrading is carried out at about 100 to about 5,000 rpm and still more preferably at about 500 to about 3,000 rpm.

The abrading of the cereal material in the present process is carried out at any temperature preferably at a temperature of about 1° C. to about 100° C. More preferably the temperature ranges from about 40° C. to about 75° C. and still more preferably, about 45° C. to about 65° C.

The present process replaces the steeping, first grind and potentially second and third grind unit operations of the conventional corn wet milling process. Additionally the present process also reduces the time typically associated with certain of these unit operations, specifically steeping. The product of the present invention can be further separated and purified into products such as starch, germ, fiber and protein using technologies associated with the conventional wet milling process.

The cereal material treated by the present method may be used for any applications where conventionally corn wet milled material has been utilized. In particular the cereal material treated by the present method is expected to be useful as fermentation feedstock. Furthermore the treated cereal material is expected to be useful in the production of fermentation feedstock, such as for example for ethanol production.

An exemplary process for carrying out the wet milling of corn treated by the present method is described as follows:

Wet processing of a cereal material may be defined as processing a cereal material wherein an amount of water exceeding the amount that can be absorbed by the cereal material is used to enhance separation of the components of the cereal material. Wet processing may entail a cereal material or a product resulting from dry grinding the cereal material. The wet processing and/or the wet milling of a cereal material will provide a product comprising starch. The corn treated by the present method is pressure fed at approximately is 6.2 bars (90 psi) through a two-pass hydrocyclone battery consisting of 15.24 cm (6 inch) hydrocyclones to separate the germ. The separated germ is washed with mill process water and dried in a rotary drum drier to yield a dried germ product which can be further processed for oil recovery. The remaining slurry from which most germ has been separated is milled again, coarsely ground using a second 91 cm (36 inch) grind mill (herewith referred as second grind) fitted with Devil's toothed plates operating at 900 rpm to detach remaining germ from ground corn in the slurry. Freed germ present in the second grind discharge slurry is separated and recovered using hydrocyclones as described above. After the removal of germ, the remaining corn material is passed over 50 micron screen (referred to as third grind dewatering screen). The filtrate containing starch-protein moves forward, while the corn material retained as overs by the screen is fine ground using a 91 cm (36 inch) grind mill (herewith referred as third grind) fitted with Devil's toothed plates operating at 1800 rpm. The fiber component in the slurry of the third grind discharge is removed by a seven stage screen separation system arranged such that the fiber is washed in a counter current flow of fiber to mill process water, where the cleanest fiber is washed with the mill process water added to the screen system. Washed fiber is discharged at the last stage (seventh stage), while starch and protein containing slurry is discharge at the first stage. The screen opening on the first fiber wash stage is 50 micron, followed by 75 micron on the second through sixth stage and 150 micron of the last stage. The washed fiber is dewatered using screw presses, and dried using a rotary drier, resulting in the dried fiber product. The discharge from the third grind dewatering screen and first stage fiber wash are combined, creating a slurry with a density of approximately 8 Baumé. This slurry is thickened with a Merco H36 centrifuge. This centrifuge operates at 2600 rpm and is fitted with No. 24 size nozzle. The overflow from the centrifuge is used as process water for steeping (also known as mill water), while the underflow slurry, having a Baumé of 12, is fed to a second H36 centrifuge (referred to as primary centrifuge). The starch-protein in the fed slurry is separated by the primary centrifuge. The primary centrifuge operates at 2200 rpm and is fitted with No. 24 nozzle to yield an underflow and overflow slurry. The overflow slurry is protein-enriched containing approximately 60% (db) protein, while the underflow slurry is starch enriched. The protein enriched overflow slurry from this centrifugation is then further dewatered by centrifugation with a third Merco H36 centrifuge operating at 2600 rpm, dewatered on a rotary drum filter and dried using a flash drier. This results in the dried protein rich product, also known as corn gluten meal. The starch enriched slurry originating from the underflow of the second Merco H36 centrifuge described above is passed through a 12 stage Dorr-Oliver clam shell hydrocyclone starch wash battery. The starch wash battery is designed such that a counter-current flow between the starch enriched stream entering the first stage of the battery and potable water entering at the twelfth stage of the battery is achieved. Each stage starch wash stage has several 10 mm hydrocyclones arranged in parallel fashion. Typical feed pressure to each starch wash stage, except the twelfth stage, is 6.2 bar (90 psi); the feed pressure on the twelfth stage is 8.27 (120 psi).

Purified starch with a slurry density of 23 Baumé is recovered as underflow from the 12 h stage of the starch wash battery, also known as starch slurry or starch product of corn wet milling.

Further information regarding the wet milling of corn is found in Technology of Corn Wet Milling and Associated Processes p. 69-125, Paul H. Blanchard, Elsevier Science Publishers B.V. Amsterdam.

The product of wet processing or wet milling comprising starch may be used in any conventional manner. For example, the product of wet processing or wet milling comprising starch may be used as a fermentation feedstock. In a further embodiment, the product of wet processing or wet milling comprising starch product may be processed into a fermentation feedstock.

As an example of a method for producing a fermentation feedstock, the following is provided. The starch comprising product produced by the previously described wet processing or wet milling processes may be optionally hydrolyzed to form a fermentation feedstock to be incorporated into the fermentation media. The starch slurry may be hydrolyzed to any extent to form a hydrolyzed starch, including to dextrose. The starch slurry may be hydrolyzed by any manner. For example, starch slurry may be hydrolyzed by subjecting the starch slurry to acid hydrolysis. Typically acids will include inorganic acids such as hydrochloric acid and the like. Elevated temperatures increase the rate of hydrolysis and may be varied over a wide range depending on the degree of hydrolysis desired. Acid hydrolysis is limited in the extent of starch hydrolysis possible. If one wishes to exceed that level of hydrolysis, one must use other means of hydrolysis such as enzymatic digestion of the starch with starch hydrolyzing enzymes.

An exemplary process for carrying out starch hydrolysis by acid hydrolysis is described as follows:
a) starch slurry with a 23 Baumé is provided;
b) the pH of the slurry is adjusted to 1.8 with 22 Baumé hydrochloric acid;
c) the slurry with pH 1.8 is introduced into a Dedert continuous acid conversion system (Olympia Fields, Ill., USA) at 146° C. (295° F.) for 18 minutes, after treatment in the conversion system the starch is hydrolyzed to 85 dextrose equivalents (DE); and
d) the pH of the converted starch is then adjusted to 4.8 with 10% soda ash and cooled.

An exemplary process for starch hydrolysis by enzyme/enzyme hydrolysis is described as follows:

Hydrolysis of starch is performed in the following two steps of 1) liquefaction and 2) saccharification.
1) Liquefaction: Water is added to the starch to adjust dry solid content to 35%. The pH of slurry is adjusted to 5.5 using sodium hydroxide solution. Calcium chloride is added to the slurry to have the minimum of 5 ppm of free calcium. TERMAMYL SUPRA enzyme, (a trademarked amylase available from Novozymes North America, Inc) is added to this pH adjusted slurry at the amount of 0.4 liter per metric ton of starch dry solids. Then, the mixture is heated in a continuous jet cooker to 108° C. (226.4° F.) and held for 5 minutes in a pressurized vessel. Then the cooked mixture is cooled to 95° C. (203° F.) and held for 100 minutes. A starch hydrolyzate with a DE of 8 to 12 is produced.
2) Saccharification: Starch hydrolyzate from the above liquefaction step is cooled to 60° C. and the dry solid content is adjusted to 32% by adding water. The pH of this diluted hydrolyzate is adjusted to 4.1-4.3 using sulfuric acid. DEXTROZYME E enzyme (a traded mixture of amyloglucosidase and pullunase available from Novozymes North America, Inc) is added at the amount of 0.7 liters per metric ton of dry solids and then the mixture is held for 40 hours.

Dextrose content of 95-97%, on the dry solid basis, is achieved.
Further information regarding starch hydrolysis is found in Technology of Corn Wet Milling and Associated Processes p. 217-266, Paul H. Blanchard, Elsevier Science Publishers B.V. Amsterdam.

The following examples are presented to illustrate the present invention and to assist one of ordinary skill in making and using the same. The examples are not intended in any way to otherwise limit the scope of the invention.

### EXAMPLES

In carrying out the examples, the following test procedure was used.

Procedure for Determining Oil Recovery:

The germ is separated from the material produced by the method of the invention by passing the material over a 1.68 millimeter (mm) opening sieve (12 Mesh). The material retained on a 1.68 mm opening sieve (12 Mesh) is floated in a 12 Baumé sodium chloride solution. The floated germ is skimmed off and germ is rinsed to remove residual sodium chloride. The separated germ is dried using Corn Refiners Association Standard Method No. G-16. The dried germ is weighed. Oil contents of the separated germ and the starting corn are determined using Corn Refiners Association Standard Method No. G-11. The oil recovery was determined as follows:
Oil recovery=[oil in germ (grams/grams of germ)×mass of germ (grams)]÷[oil in starting corn (grams/grams of corn used)×mass of corn used (grams)]×100

### Example 1

Example 1 illustrates a method for processing cereal material wherein 150 grams of corn is continuously and simultaneously both absorbing solvent and abraded. In carrying out the runs a Waring blender (Model 51BL32, VWR International, Indianapolis, Ind.) equipped with a 1-Liter stainless steel container with the standard stainless steel blades inverted was utilized. The blender is equipped with a tachometer (Dent Controls, model DM4004) to monitor the revolutions per minute (rpm) of the blades and a variable transformer for control of the revolutions per minute of the blade. In carrying out the runs, the corn and the solvent are simultaneously introduced into the blender. The details of each of the experimental runs, and the oil recovery, are reported in Table 1.

TABLE 1

| | | Solvent | | | | |
|---|---|---|---|---|---|---|
| | | absorption and | Abrading | | Total | |
| Run | | abrading time | speed | Temperature | solvent | Oil recovery |
| No. | Solvent Type | (min) | (RPM) | (° C.) | mass (g) | (%) |
| 1 | Steep solution¹ | 60 | 1600 | 50 | 400 | 50.8 |
| 2 | Steep solution¹ | 90 | 1200 | 50 | 400 | 77.2 |
| 3 | Steep solution¹ | 90 | 1600 | 50 | 400 | 67.3 |
| 4 | Steep solution¹ | 90 | 1600 | 50 | 250 | 62.7 |
| 5 | Steep solution¹ | 90 | 1600 | 50 | 550 | 68.0 |
| 6 | Steep solution¹ | 90 | 1600 | 22 | 400 | 62.2 |
| 7 | Steep solution¹ | 90 | 2000 | 50 | 400 | 62.8 |
| 8 | Steep solution¹ | 120 | 1600 | 50 | 400 | 81.0 |
| 9 | Water | 90 | 1600 | 50 | 400 | 60.5 |

¹The steep solution was an aqueous solution containing 0.2% (w/w) of sulfur dioxide and 1.0% (w/w) lactic acid

### Example 2

Example 2 illustrates a method for processing cereal material, wherein the cereal material, 150 grams of corn, was presoaked in solvent and the resulting presoaked corn was continuously and simultaneously both absorbing solvent and being abraded. In this example, the subsequent absorption was achieved using the residual solvent from the presoak treatment.

The solvent used for presoak and subsequent absorption was an aqueous solution containing 0.2% (w/w) of sulfur dioxide and 1.0% (w/w) lactic acid. The amount of solvent used during these runs was about 400 grams.

The presoaked corn and the residual solvent were introduced into a Waring Blender (Model 51BL32, VWR International, Indianapolis, Ind.) equipped with a 1-Liter stainless steel container with standard stainless steel blades inverted. The blender is equipped with a tachometer (Dent Controls, model DM4004) to monitor the revolutions per minute (rpm) of the blades and a variable transformer for control of the revolutions per minute of the blades.

In the runs of example 2, the abrading rpm (revolutions per minute) was increased over the time of the run. The time of the run was 90 minutes after the presoak time. The abrading rpm was 1,200 rpm at start, 1,300 rpm at 15 minutes, 1,400 rpm at 30 minutes, 1,500 rpm at 50 minutes, 1,700 rpm at 60 minutes and 2,000 rpm at 75 minutes. The temperature was maintained at 50° C. for the entire 90 minutes.

The details of each of the experimental runs and oil recovery are reported in Table 2.

TABLE 2

| | Pre-Soak time | |
|---|---|---|
| Run No. | (Minutes) | Oil recovery (%) |
| 1 | 15 | 69.3 |
| 2 | 30 | 67.8 |
| 3 | 60 | 65.1 |
| 4 | 960 | 66.8 |

### Example 3

Example 3 illustrates a method for processing cereal material, wherein the cereal material, corn, and the solvent are introduced into a 2-liter cylinder. The corn and the solvent are constantly agitated using dispersion blades (Catalogue No. 14215-318, VWR International Inc, USA) during the course of the run. A a portion of the the corn and the solvent is continuously introduced into a Quaker city mill (Model 4E, The Straub Co., Philadelphia, Pa., USA). The clearance of the grinding plates is slightly less than the average corn thickness. After passing through the mill the slip stream is returned to the cylinder with the agitator. The total duration of the run is about 90 minutes during which time the temperature was maintained at about 50° C.

The solvent used is an aqueous solution containing 0.2% (w/w) of sulfur dioxide and 1.0% (w/w) lactic acid. The amount of solvent used during this run is about 400 grams.

In the run of example 3 the abrading plate clearance of the Quaker city mill (Model 4E, The Straub Co., Philadelphia, Pa., USA) is decreased over the time of the run. The abrading plate clearance is 5.08 mm for the first 20 minutes, 4.572 mm for next 20 minutes, 3.048 mm for next 20 minutes, 1.525 mm for next 20 minutes and 0.762 mm for the next 10 minutes.

### Example 4

The procedure of example 1 is followed except that barley is substituted for the corn. It is expected that similar results will be obtained.

### Example 5

The procedure of example 1 is followed except that dispersion blades (Catalogue No. 14215-318, VWR International Inc, USA) was used at an rpm of 3,000. It is expected that similar results will be obtained.

### Example 6

The procedure of example 1 is followed except that the temperature is maintained at 60° C. It is expected that similar oil recovery results will be obtained.

### Example 7

The procedure of example 3 is followed except that instead of the Quaker City mill, a Waring Blender (Model 51BL32, VWR International, Indianapolis, Ind.) equipped with a 1-liter stainless steel container with the standard stainless steel blades inverted was utilized. The blender is equipped with a tachometer (Dent Controls, model DM4004) to monitor the revolutions per minute (rpm) of the blades and a variable transformer for control of the revolutions per minute of the blades. The blade rpm was set at 1200 for the entire duration. It is expected that similar results will be obtained.

From the above examples it is observed that the present method results in cereal material being produced in a shorter period of time and with less number of unit operations, than conventional processes. For example, the use of dry milling processes while requiring few unit operations typical have oil recovery of only about 30 to about 55%. Use of conventional wet milling processes, can produce oil recoveries of 65-85%. However, this is achieved at the cost of long steeping times of 24-50 hours.

The invention has been described with reference to various specific and illustrative embodiments and techniques. However, one skilled in the art will recognize that many variations and modifications may be made while remaining within the sprit and scope of the invention.

1. A method for processing a cereal material comprising providing a cereal material, and continuously and simultaneously both having solvent absorbed by the cereal material and abrading the cereal material for a period of at least about 1 minute

2. The method according to claim 1 wherein the cereal material is abraded at about 5 to about 10,000 revolutions per minute.

3. The method according to claim 2 wherein the cereal material is abraded at about 100 to about 5,000 revolutions per minute.

4. The method according to claim 2 wherein the cereal material is abraded at about 500 to about 3,000 revolutions per minute.

5. The method according to claim 1 wherein the period of absorption of the solvent and abrasion of cereal material ranges from about 1 hour to about 3 hours.

6. The method according to claim 1 wherein the temperature ranges from about 1° C. to about 100° C.

7. The method according to claim 6 wherein the temperature ranges from about 45° C. to about 65° C.

8. The method according to claim 1 wherein the cereal material is selected from the group consisting of corn, oats, barley, wheat, rice, sorghum and mixtures thereof.

9. The method according to claim 1 wherein the solvent absorbed by the cereal material is a solvent selected from the group consisting of an aqueous solution, an organic solution and mixtures thereof.

10. The method according to claim 9 wherein the solvent further comprises at least one compound selected from the group consisting of wetting agents, reducing agents, enzymes, and pH modifiers.

11. The method according to claim 9 wherein the solvent is water.

12. The method according to claim 1 further comprising separating germ, fiber and protein from processed cereal material to provide a starch containing stream.

13. The method according to claim 12 wherein the starch containing stream is hydrolyzed.

14. A method for producing a fermentation feedstock comprising using the starch containing stream produced in accordance with claim 12.

15. A method for producing a fermentation feedstock comprising using the hydrolyzed starch containing stream produced in accordance with claim 13.

16. A method for using the starch containing stream of claim 12 as fermentation feedstock.

17. A method for using the hydrolyzed starch containing stream of claim 13 as fermentation feedstock.
